(19) United States
(12) Patent Application Publication
(10) Pub. No.: US 2003/0232100 A1
(43) Pub. Date: Dec. 18, 2003

(54) Compositions for treatment of diseases arising from secretion of mast cell biochemicals

(76) Inventor: Theoharis C. Theoharides, Brookline, MA (US)

Correspondence Address: Dr. Melvin Blecher, Law Offices of Dr. Melvin Blecher, 4329 Van Ness St., NW, Washington, DC, 20016, US

(21) Appl. No.: 10/439,301

(22) Filed: May 16, 2003

Related U.S. Application Data
(63) Continuation-in-part of application No. 09/773,576, filed on Feb. 2, 2001.
Application No. 09/773,576 is a division of application No. 09/056,707, filed on Apr. 8, 1998.

(51) Int. Cl. A61K 35/78; A61K 31/737; A61K 31/728; A61K 31/7048; A61K 31/7008; A61K 31/522; A61K 31/353; A61K 31/202; A61K 31/13

(52) U.S. Cl. 424/769; 514/27; 514/54; 514/62; 424/771; 514/263.31; 514/560; 514/46; 514/456; 514/674

(57) ABSTRACT

Compositions for treatment of diseases arising from products secreted by activated tissue mast cells, composed of, as active ingredients, unprocessed olive kernel (pit) extract that increases absorption of these compositions in various routes of administration, and one or more of a heavily sulfated, non-bovine proteoglycan such as shark cartilage chondroitin sulfate C, a hexosamine sulfate such as D-glucosamine sulfate, a flavonoid such as quercetin, S-adenosylmethionine, a histamine-1 receptor antagonist, a histamine-3 receptor agonist, a CRH antagonist, caffeine, fragments of myelin basic protein, rutin, polyunsaturated fatty acids, Bitter Willow Extract and a polyamine.

This application is a continuation-in-part of co-pending U.S. Ser. No. 09/773,576, filed Feb. 2, 2001, which is a divisional of co-pending U.S. Ser. No. 09/056,707, filed Apr. 8, 1998.

### BACKGROUND OF THE INVENTION

The invention generally relates to the treatment of diseases arising from mast cell secretory products. More specifically, the invention relates to compositions containing inhibitors of mast cell activation and secretion that are designed to be used as dietary supplements alone or as or adjuvants to conventional approved medications for the relief of said diseases. The expression “arising from” is intended herein to mean any process that leads to pathophysiology that involves any product secreted from mast cells. The term “secretory product” is intended to mean any biochemical(s) secreted from mast cells, whether preformed or newly synthesized. By “disease” is mean any condition, syndrome or other pathophysiological entity leading to dysfunction in the patient.

A recent meta-analysis showed potential therapeutic benefit of chondroitin sulfate and/or glucosamine in osteoarthritis [McAlindon et al. J Am Med Assn. 283:1469 (2000)], while a double-blind clinical trial with glucosamine showed definite benefits in osteoarthritis with respect to pain, radiographic joint appearance and progression [Reginster et al., Lancet 337:252 (2001); Pavelka et al., Arch Intern Med. 162:2113(2002)]. However, less than 5% of the chondroitin sulfate in commercially available preparations is absorbed orally, because the size of the molecule and the degree of sulfation impede its absorption from the gastrointestinal tract, which greatly reduces the effectiveness of such preparations. Furthermore, such commercial preparations use chondroitin sulfate obtained from cow trachea, with the possible danger of contracting spongiform encephalopathy or “mad cow disease”. In fact, the European Union has banned even cosmetics that contain bovine-derived products.

Theoharides et al., British Journal of Pharmacology 131:1039 (2000) indicated for the first time how proteoglycans such as chondroitin sulfate may work. This paper reported that chondroitin sulfate and, to a lesser degree, glucosamine sulfate, inhibit activation of mast cells that are known to trigger allergy and asthma, and initiate inflammation. This discovery was the basis for Theoharides, U.S. patent applications Ser. No. 09/056,707, filed Apr. 8, 1998 and Ser. No. 09/773,576, filed Feb. 2, 2001, which are incorporated herein by reference.

Mast cells are known to be causative for allergic conditions, and are also now recognized as important causative intermediary in many painflul inflammatory conditions [Galli, N Eng J Med. 328:257 (1993); Theoharides, Int J Tissue Reactions 18:1 (1996)], such as interstitial cystitis and irritable bowel syndrome [Theoharides, Ann NY Acad, Sci. 840:619 (1998)], as well as in migraines and possibly multiple sclerosis [Theoharides, Persp Biol Med. 26:672 (1983); Theoharides, Life Sci 46:607 (1996)].

Mast cells increasingly implicated in conditions involving inflamed joints, such as in osteoarthritis and rheumatoid arthritis, through activation of local mast cells by, for example, neuropeptides, such as Substance P [Theoharides, T C, et al. Eur. J. Inflamm 1:1 (2003). Moreover, it is now known that activation of uterine mast cells may lead to spontaneous abortion [Madhappan et al., Endocrinology (2003). In addition, human mast cells release the cytokine IL-6 which has been linked to bone resorption and osteoporosis [Theoharides, T C et al., J. Clin. Psychopharm. 13:1(2003).

It was recently shown that chondroitin sulfate's ability to inhibit the activation of mast cells compliments the inhibitory effects on mast cell activation of another class of naturally occurring compounds, the flavonoids [Middleton et al. Pharm Rev 52:1 (2000)]. Certain plant flavones (in citrus fruit pulp, seeds, sea weed) are now recognized as anti-allergic, anti-inflammatory, anti-oxidant and cytoprotective with possible anti-cancer properties. However, only some flavonoids belonging to the subclass of flavones, e.g., quercetin, inhibit mast cell activation.

Quercetin inhibits secretion from human activated mast cells [Kimata et al. Allergy 30:501(2000)], and has also been used effectively for the treatment of chronic prostatitis [Shoskes et al., Urology 54:960 (1999)]. However, other flavonoids may have opposite effects. Use of the term “bioflavonoids” or “citrus flavonoids” in certain commercial products, therefore, provides little information, and may include molecules that have detrimental effects; for example, soy contains isoflavones that have estrogen-like activity that worsens inflammatory conditions.

Copending U.S. patent applications Ser. No. 09/056,707, filed Apr. 8, 1998, and divisional Ser. No. 09/773,576, which are incorporated herein by reference, claim the oral use of proteoglycans, without and with flavonoids, for the treatment of mast cell activation-induced diseases. However, issues involving absorption of these compositions from the gastrointestinal tract and synergism with other treatment modalities were not addressed in these applications.

Applicant has described the use of antagonists of the actions of Corticotropin Releasing Hormone (CRH), also known as Corticotropin Releasing Factor(CRF). The applicant has proposed that CRH and its structural analogue Urocortin (Ucn) can stimulate mast cells in various tissues, leading to inflammatory disease. CRH antagonists inhibit myocardial mast cell activation in myocardial ischemia (copending U.S. patent application Ser. No. 08/858,136, filed May 18, 1997), in treating stress-induced skin disease (U.S. Pat. No. 6,020,305), and in treating stress-induced migraine headaches (U.S. Pat. No. 5,855,884), the contents of which are incorporated herein by reference. The synergistic effects of the compositions of the present invention, that include antagonists of the actions of CRH on mast cells, were not recognized at the time of the previous studies, nor were problems associated with the absorption of these compositions from the gastrointestinal tract, lungs and skin. The word “antagonists” in connection with CRH is intended herein to include any molecule that prevents the actions of CRH on target cells, and includes, but is not limited to, anti-CRH neutralizing antibodies or binding proteins, or molecules preventing the release of CRH at local sites and CRH receptor antagonists (see below for details).

Applicant has also described a method for treating interstitial cystitis patients induced by activated mast cell derived molecules with histamine-1 receptor antagonists (Theoharides, U.S. Pat. No. 5,994,357, incorporated herein by reference). Treatment of mast cell-induced migraines with histamine-1 receptor antagonists is the subject of Theoharides, U.S. Pat. No. 5,855,884, which is incorporated herein by reference. Histamine-3 receptor agonists as pharmaceutical agents in mast cell-involved diseases are described in Theoharides U.S. Pat. No. 5,831,259, which is incorporated herein by reference. At the time of this invention the synergistic effects of the present compositions with such antagonists, and absorption problems had not yet been recognized.

An important need therefore exists for compositions for administration to patients being treated for diseases arising from mast cell secretory products by various modalities, that are synergistic in that they have stronger effects than the sum of the effects of the individual components, and are synergistic with conventional clinical treatments of inflammatory conditions, “Synergistic” is also intended to mean: “coordinated or correlated action by two or more structures or drugs” [Stedman's Medical Dictionary, 23rd edition, Williams & Wilkins, Baltimore, 1976]. An important need also exists for formulations that increase the absorption from the gastrointestinal tract, nasal passages, mouth, eye and skin surface of the compositions of the invention. Such formulations have been discovered, and are described below.

### SUMMARY OF THE INVENTION

The invention comprises compositions for treatment of diseases arising from mast cell secretory products, containing an unprocessed olive kernel (pit) extract (“OKE”), and one or more active ingredients selected from the group consisting of a non-bovine sulfated proteoglycan, a hyaluronan, a sulfated hexosamine, a flavonoid compound, S-adenosylmethionine (“SAMe”), histamine-1 receptor antagonists, histamine-3 receptor agonists, antagonists of the actions of CRH, caffeine, folic acid, rutin, polyunsaturated fatty acids especially eicosapentenoic acid (“EPA”), fragments of myelin basic protein, Bitter Willow Extract and polyamines, together with appropriate excipients, carriers, plant extracts, flavorings and fragrances, said compositions having improved absorption from the gastrointestinal tract, skin surface, nasal, oral, ocular and pulmonary surfaces, and effects that are synergistic with each other and synergistic with available conventional clinical treatment modalities.

Diseases treatable by the inventive compositions include: allergic inflammation, arthritis (including osteoarthritis and rheumatoid arthritis), asthma, endometriosis, fibromyalgia, inflammatory bowel disease, interstitial cystitis, irritable bowel syndrome, migraines, cardiovascular diseases, coronary artery diseases, chronic prostatitis, eczema, multiple sclerosis, psoriasis, sun burn, vitiligo, alopecia, periodontal diseases, superficial vasodilator (flush) syndromes, recurrent spontaneous chronic pregnancy loss (miscarriages), hormonally-dependent cancers, chemotherapy-associated oral mucosa damage.

In one embodiment, the sulfated glucosamine is D-glucosamine sulfate, the proteoglycan is non-bovine chondroitin sulfate, the flavonois is quercetin, and the factor increasing absorption of the composition is an extract of olive kernels (OKE).

In another embodiment, compositions may also contain antagonists of the effects of CRH on mast cells or other target cells of the myocardium, gastric mucosa, ocular, oral and pulmonary mucose, urinary bladder, prostate, myometrium, endometrium, placenta, skin, meningeal membranes, and blood-brain barrier.

In still another embodiment, the present compositions are used against superficial vasodilator flush syndromes, such as those associated with carcinoid syndrome and ingestion of niacin.

in yet another embodiment, the present compositions are used against recurrent spontaneous pregnancy loss.

### DETAILED DESCRIPTION OF THE PREFERRED EMBODIMENTS OF THE INVENTION

It has been discovered that a combination of a unique unprocessed olive kernel (pit) extract (“OKE”), together with one or more of a non-bovine sulfated proteoglycan, a hyaluran, a sulfated D-hexoseamine, a flavonoid, CRH and Ucn antagonists, histamine-1 receptor antagonists, histamine-3 receptor agonists, fragments of myelin basic protein, polyunsaturated fatty acid, polyamines and caffeine has synergistic anti-inflammatory effects when used as a dietary supplement, a topical product or an aerosol for nasal, ocular, oral and skin surfaces or pulmonary administration, without or with a conventional clinical treatment for inflammatory diseases. As used herein, “inflammatory diseases” is to be taken as meaning inflammatory diseases resulting from the activation, degranulation and consequent secretion of inflammatory biochemicals from mast cells, and the resultant inflammatory diseases include the group consisting of: allergic inflammation, arthritis (to include osteoarthritis and rheumatoid arthritis), asthma, endometriosis, fibromyalgia, inflammatory bowel disease, interstitial cystitis, irritable bowel syndrome, migraines, vitiligo, alopecia, cardiovascular disease, coronary artery disease, chronic prostatitis, eczema, multiple sclerosis, psoriasis, sun burn, periodontal disease of the gums, superficial vasodilator (flush} syndromes, recurrent spontaneous pregnancy loss, hormonally-dependent cancers, and chemotherapy-associated oral mucosa damage.

The OKE component of the inventive compositions is preferably an unprocessed (first pressing, filtered, oleic acid-related acidity <3%, water content <5%) extract produced, for one source, on the island of Crete in Greece. This OKE extract is especially prepared by the maker by a process consisting essentially of: (1) washing with water the kernel mass that remains after the compression of the oil from the olive flesh, branches and leaves (this mass is called Sansa in the Greek language); (2) drying the washed kernels in a stream of hot air at about 80° C. to reduce the water content to about 5%; (3) extracting the dried kernels with hexane and steam; (4) cooling the hexane extract and microfiltering the extract (5 micron pore size) to remove particulate matter; (5) heating the hexane extract at about 40° C. degrees without or with percolating helium (to avoid oxidation) through the fluid to evaporate the hexane (final <0.5%), which process further reduces the water content to <1% and the acidity (as oleic acid) to <3% ; and (6) storing the extract in sealed containers. While hexane is preferred as the organic solvent, other similar organic solvents, such as are known by others skilled in this art, are also suitable.

When this OKE is mixed with the powdered active ingredients of the inventive compositions it forms lipid micelles (liposomes) that surprisingly have the unique property of increasing absorption of the other components of the anti-inflammatory compositions through the intestinal mucosa, skin and other body surfaces (see Example 18 below that provides in vivo evidence for this property). OKE also contributes its own content of important anti-oxidants [Bosku, World Rev Nutr Diet, 87:56 (2000)], such as omega fatty acids (e.g., eicosapentanoic acid) and α-tocopherol. The polyphenols in such kernel extracts also have anti-inflammatory effects in, for example, arthritis [Martinez-Dominguez et al., Inflamm. Res. 50:102 (2001)]. A preferred source of the OKE of the invention is: E.B.E.K., Inc., Commercial, Industrial Enterprises of Crete, 118 Ethnikis Antistasecos, Heraklion, Crete, 71306, Greece.

In a highly preferred embodiment, the sulfated proteoglycan is non-bovine chondroitin sulfate, most preferably derived from shark cartilage, which blocks mast cell activation, degranulation and consequent secretion of inflammatory biochemicals from the mast cells. Other natural sulfated proteoglycans suitable for practicing this invention include keratan sulfate, dermatan sulfate and a hyaluronan such as sodium hyaluronate. The chondroitin sulfate from shark cartilage is more-highly sulfated than the common commercial chondroitin sulfate isolated from cow trachea; the shark cartilage source also avoids the potential dangers associated with bovine sources, such as spongioform encephalopathy (“mad cow disease”).

The highly preferred flavone is quercetin which inhibits secretion of inflammatory molecules from mast cells by inducing phosphorylation of moesin, a unique 78 kDa mast cell protein [Theoharides et al. J Pharm Exp Therap 294:810 (2000)]. In addition to quercetin, other flavones suitable in carrying out the invention include myricetin, genistein, kaempferol and the quercetin glycoside rutin. A highly preferred source of quercetin and its glycoside is the Saphora plant.

Supplementation of the compositions described above with the methylation reagent S-adenosylmethionine (SAMe) increases the antioxidant, anti-inflammatory and cytoprotective properties, particularly in inflammatory joint diseases. Addition of SAMe also accelerates metabolism of homocysteine, which amino acid has been implicated in coronary artery disease, to cysteine, which is harmless. Folic acid may be added to certain of the present formulations for similar reasons.

Another supplement to the basic compositions of the invention is a particular histamine-1 receptor antagonist, such as hydroxyzine, azelastine, azatadine and cyproheptadine. Other suitable histamine-1 receptor are described in the Theoharides patents listed above and incorporated by reference. Effective histamine-3 receptor agonists are also described in the Theoharides patents listed above and incorporated by reference.

Addition of Bitter Willow Bark Extract {which contains salicylic acid) to the inventive compositions adds anti-inflammatory effects.

Inhibitors of mast cell activation and secretion, especially serotonin, may be used in the treatment of inflammatory processes such as superficial vasodilator syndrome, e.g., menopausal-associated flush, monosodium glutamate-associated flush, carcinoid flush and niacin-associated flush.

Sources of suitable CRH antagonists include, in addition to those listed in the Theoharides patents described above: Neurocrine Biochem. Inc.'s (La Jolla, Calif.) D-Phe 12 Nle Ala32,21,38hCRH(12-41)NH2, cat no. 1P-36-41; Pfizer (Groton, Conn.) non-peptide CP-154,526-1; Sigma Chem. Co., St. Louis anti-CRH polyclonal antiserum; Pfizer patents and applications: U.S. Pat. No. 6,211,195, U.S. Pat. No. 5,795,905, PCT/IB95/00573, PCT/IB95100439, U.S. Ser. No. 08/448,539, U.S. Ser. No. 08/481,413, U.S. Ser. No. 09/735,841, and Owens et al. Pharm. Rev. 43:425 (1991), all of which are incorporated by reference.

The preferred concentration range of the proteoglycan, hexosamine sulfate and flavonoid components of the oral formulations are 100-3,000 mg per capsule. The preferred concentration range for SAMe is 3-1,000 mg per capsule. Generally, the amounts of OKE are at least 2-3 times those of the other active ingredients, preferably 100-1500 mg, although lower amounts can also be used effectively. The number of capsules to be taken per day is determined by the nature and severity of the disease, and is readily determinable by the patient's health provider. Other representative formulations are described in the examples below.

The compositions of the invention may be formulated in any standard means of introducing pharmaceuticals into a patient, e.g., by means of oral capsules. The compositions of the invention may also include ointments and creams for skin diseases, mouth washes, oral gels and toothpaste for periodontal diseases, as well as solutions for nasal aerosols and eye drops, as well intravaginal suppositories. Standard excipients and carriers for the active ingredients of the inventive compositions are described in Remington's Pharmaceutical Sciences, Mack Publishing Co., Easton, Pa. Fragrances, flavorings and vitamins and mineral salts, e.g., KCl, may also be added, as appropriate.

Although not bound by any particular mechanism of action of the components of the claimed compositions, the inventor contemplates that the proteoglycan inhibits the activation and degranulation of the relevant mast cells, while the flavone inhibits the secretion of inflammatory biomolecules from these mast cells. The inhibition of two distinct steps in a pathway leading to secretion of said biomolecules constitutes synergism, as is well known in the art. “Activation” and “degranulation” of mast cells are defined herein as is standard and well known in this art, that is, to mean secretion from the activated mast cell of any type of molecule(s) that alone or in combination triggers inflammatory processes.

### EXAMPLES

### Example 1

Table 1 compares chondroitin-containing commercial products to the present compositions.

TABLE 1
Comparison of Chondroitin Sulfate-Containing Products
to Present Invention

| | Most Available | |
|---|---|---|
| Product | Compositions | Present Invention |
| Main ingredient | Mixture of | Non-bovine chondroitin |
| | chondroitins | sulfate, preferably the C |
| | | type |
| Source | Cow trachea | Shark cartilage |
| Amount per | 100-300 | 10-3000 mg |
| capsule or tablet | | |
| Degree of | Low, if any | High |
| sulfation | | |
| Absorption from | <5% | >15% |
| g.i. tract | | |
| Target | Unknown | Mast cells, inflammatory |
| | | cells |
| Carrier | Powder | OKE |
| Other ingredients | Vitamins, fish oils | Flavones, SAMe, histamine- |
| | | 1 receptor antagonists, |
| | | histamine-3 receptor |
| | | agonists, CRH antagonists, |
| | | polyamines, caffeine, folic |
| | | acid, bitter willow extract. |
| Advantages | Presumably | Anti-allergic, anti- |
| | cartilage | inflammatory, anti-oxidant, |
| | synthesis | cytoprotective |
| Adverse effects | Risk of spongiform | None known |
| | encephalopathy, | |
| | stomach upset, | |
| | diarrhea | |
| Relevant | Osteoarthritis | Arthritis (osteoarthritis or |
| conditions | | rheumatoid arthritis), |
| | | allergic inflammation |
| | | angina, asthma coronary |
| | | artery disease, chronic |
| | | prostatitis, eczema, |
| | | fibromyalgia, interstitial |
| | | cystitis, irritable bowel |
| | | syndrome, inflammatory |
| | | bowel disease, migraines, |
| | | multiple sclerosis, psoriasis, |
| | | periodontal disease, flush |
| | | syndrome, cancer (especially |
| | | hormonally-dependent |
| | | forms), recurrent |
| | | spontaneous pregnancy loss . . . |
| Scientific | None found | Theoharides et al. Br J |
| publications | | Pharm 131:1039 (2000) |
| | | Middleton et al. Pharin Rev |
| | | 52:673 (2000) |

In all examples and claims, chondroitin sulfate is to be assumed to be of a non-bovine variety.

### Example 2

Composition For Protecting Against Inflammatory Diseases)
Two capsules to be taken orally 2-3 times daily,
at least one hour before meals

| Ingredients, per capsule, | mg: |
|---|---|
| OKE | 500-1200 |
| Chondroitin sulfate | 150-300 |
| D-Glucosamine sulfate | 150-300 |
| Quercetin | 150-300 |

### Example 3

Composition For Protecting Against Arthritis

| Ingredients per capsule, | mg: |
|---|---|
| OKE | 500-1200 |
| D-Glucosamine sulfate | 150-300 |
| Chondroitin sulfate | 150-300 |
| Sodium hyaluronate | 100-200 |
| Quercetin | 150-300 |

### Example 4

Topical Composition For Protecting Against Arthritis
Skin ointment or cream. Apply three times per day
to affected areas.

| Ingredients | % by weight |
|---|---|
| OKE | 15-50 |
| Aloe extract | 5 |
| Chondroitin sulfate | 5 |
| Sodium hyaluronate | 5 |
| Bitter willow bark extract | 5 |
| Quercetin | 3 |

### Example 5

Composition For Protecting Against Cardiovascular Disease
Two capsules to be taken orally 2-3 times per day, in mg:

| | |
|---|---|
| OKE | 500-1200 |
| Chondroitin sulfate | 50 |
| Quersetin | 100 |
| S-adenosylmethionine | 50 |
| Niacin | 100 |
| Folic acid | 20 |
| EPA | 200-500 |
| Bitter willow bark extract | 5% by weight |

### Example 6

Composition For Protecting Against Periodontal Disease

| Mouthwash: | | |
|---|---|---|
| OKE | 500-1200 | mg |
| Chondroitin sulfate | 0.3-0.4 | M |
| Quercetin | 0.3-0.4 | M |
| optionaIly, D-glucosamine, 0.4 M and SAMe | 0.15 | M |
| In a standard mouthwash vehicle | | |

### Example 7

Toothpaste Composition

| Toothpaste, | mg %: |
|---|---|
| OKE | 30-50 |
| Chondroitin sulfate | 5 |
| Quercetin | 3 |
| optionally D-glucosamine | 5 |
| Optionally, fluoride | |
| In a standard toothpaste vehicle | |

### Example 8

Sunscreen composition

| Ingredients | mg % |
|---|---|
| OKE | 30-50 |
| Chondroitin sulfate | 5 |
| Aloe extract | 5 |
| Quercetin | 3 |
| Optionally, azelastine or hydroxyzine | 5 |
| Optionally, D-glucosamine | 5 |
| Sun screen (e.g., TiO₂) | 5 |

### Example 9

Composition For Protecting Against Migraine Headaches

| Ingredients, | mg: |
|---|---|
| OKE | 500-1200 |
| Chondroitin sulfate | 50 |
| Quercetin | 100 |
| Azatadine | 4 |
| Caffeine | 20 |
| Optionally, a CRH-receptor antagonist | |

### Example 10

Composition For Protecting Against Relapsing
Multiple Sclerosis

| Ingredients, | mg: |
|---|---|
| OKE | 500-1200 |
| Chondroitin sulfate | 50 |
| Quercetin | 400 |
| Hydroxyzine | 50 |
| Caffeine | 20 |
| Optionally, interferon-beta | |

### Example 11

Composition For Protecting Against Cystitis And Prostatitis

| Ingredients, | mg: |
|---|---|
| OKE | 500-1200 |
| D-glucosamine sulfate | 50-150 |
| Chondroitin sulfate | 100-300 |
| Sodium hyaluronate | 20 |
| Quercetin | 100-400 |
| Optionally, rutin | |

### Example 12

Composition For Protecting Against “Flush”

| Ingredients, per capsule: | |
|---|---|
| OKE | 500-1200 |
| Chondroitin sulfate | 50 mg |
| Quercetin | 150 mg |
| Bitter willow bark extract | 5% by weight |
| Optionally, cyproheptadine or azatadine | 4 mg |

### Example 13

Cream Composition For Protecting Against Skin Allergy

| Ingredients: | % by weight |
|---|---|
| OKE | 15-50 |
| Aloe vera | 5 |
| Chondroitin sulfate | 5 |
| Myricetin | 5 |
| Alpha-tocopherol | 5 |
| Optionally, azelastine or hydroxyzine | 5 |

### Example 14

Composition For Protecting Against Allergy and Allergic Asthma

| Ingredients, | mg |
|---|---|
| OKE | 500-1200 |
| Myricetin | 500 |
| Chondroitin sulfate | 200 |
| Optionally, azelastine or hydroxyzine | 50 |

### Example 15

Composition For Protecting Against
Hormonally-Dependent Cancers

| Ingredients, | mg |
|---|---|
| OKE | 500-1200 |
| Chondroitin sulfate | 200 |
| Quercetin | 150 |
| Genestein | 50 |

### Example 16

Composition For Protecting Against
Allergic Conjunctivitis or Rhinitis

| Ingredients | w/v |
|---|---|
| OKE | 15-50% |
| Quercetin | 0.05% |
| Chondroitin sulfate | 2.0%\ |
| Optionally, azelastine | 0.05% |

### Example 17

Intravaginal Composition for Protecting Against
Spontaneous Abortion

| Ingredients | mg |
|---|---|
| OKE | 500-1200 |
| Chondroitin sulfate | 200 |
| Quercetin | 500 |
| Optionally, azelastine | 10-50 |

### Example 18

Effect of Olive Kernel Extract on Absorption of Proteoglycan Sulfate In Vivo

Chondroitin sulfate was tritiated by New England Nuclear to a specific activity of 4.3 mCi/ml.

2.5 mCi of tritiated chondroitin sulfate was given orally to 250 g laboratory rats without (control) and with (experimental) OKE. Serum radioactivity was measured 8 hours thereafter.

The results showed that, in control animals, about 3.9% of the dose reached the circulation. In sharp contrast, in animals given OKE along with the labeled chondroitin sulfate, about 14.3% of the dose was absorbed into the general circulation.

The results showed that, at 8 hours post-adminsitration, OKE had increased by almost 400% the absorption of the proteoglycan from the intestine into the circulation of these animals.

These examples should not be interpreted as limiting the scope of the claims, which is limited only by the recitations of the claims themselves.

I claim:

1. A composition comprising, as active ingredients, unprocessed olive kernel extract (“OKE”) and one or more of a non-bovine proteoglycan sulfate, a hexosamine sulfate, a flavonoid compound, S-adenosylmethionine (“SAMe”), a histamine-1 receptor antagonist, a histamine-3 receptor agonist, an antagonist of the actions of CRH or Ucn, a hyaluronan, Bitter Willow Extract, a polyunsaturated fatty acid, a polyamine, rutin, myelin basic protein fragment, and caffeine, in an appropriate excipient or vehicle containing optional fragrances, colorants, vitamins, mineral salts and plant extracts, wherein said composition exhibits the property of preventing or reversing diseases arising from the activation of mast cells, consequent degranulation of said cells, and consequent secretion of disease-causing biomolecules.

2. The composition according to claim 1, wherein said OKE exhibits an acidity as oleic acid of <3%.

3. The composition according to claim 1, wherein said non-bovine sulfated proteoglycan is selected from the group consisting of non-bovine chondroitin sulfate, keratan sulfate, dermatan sulfate and sodium hyaluronate.

4. The composition according to claim 2, wherein said non-bovine chondroitin sulfate is chondroitin sulfate C derived from shark cartilage.

5. The composition according to claim 1, wherein said hexosamine sulfate is D-glucosamine sulfate.

5. The composition according to claim 1, wherein said flavone is selected from the group consisting of quercetin, myricetin, genistein and kaempferol.

7. The composition according to claim 1, said composition being for oral use, comprising 500-1200 mg of OKE, and 10-3,000 mg per capsule of each of chondroitin sulfate C, quercetin and D-glucosamine sulfate.

8. The composition according to claim 7, further supplemented with 3-1,000 mg of SAMe per capsule.

9. A composition according to claim 1, wherein said diseases are selected from the group consisting of: arthritis, fibromyalgia, inflammatory bowel disease, interstitial cystitis, irritable bowel syndrome, migraines, asthma coronary artery disease, cardiovascular disease, chronic prostatitis, eczema, multiple sclerosis, psoriasis, sun burn, tooth decay, periodontal disease, chemotherapy or radiation-induced oral mucosa damage, migraines, opening of bladder mucosa, opening of the blood-brain barrier, spontaneous recurrent pregnancy loss, superficial vasodilator(flush} syndrome, and hormonally-dependent cancers.

10. The composition according to claim 9, wherein said disease is arthritis and said composition is for oral administration, comprising OKE, chondroitin sulfate, quercetin, D-glucosamine sulfate, and, optionally, sodium hyaluronate, eicosapentenoic acid (“EPA”), and Bitter Willow Extract.

11. The composition according to claim 9, wherein said disease is arthritis and said composition is for topical use, said composition further comprising D-glucosamine sulfate,, sodium hyaluronate, Bitter Willow Extract, and quercetin.

12. The composition according to claim 9 for oral or aerosol use in allergic conditions, said composition further comprising a flavonoid selected from the group consisting of quercetin, genestein and, optionally, a histamine-1 receptor antagonist.

13. The composition according to claim 9, for topical use in allergic conditions, said composition further comprising, myricetin, alpha-tocopherol, and, optionally, a histamine-1-receptor antagonist.

14. The composition according to claim 13, wherein said histamine-1 receptor antagonist is diphenylhydramine, hydroxyzine, azatadine, azelastine or cyproheptadine.

15. The composition according to claim 9 wherein said inflammatory disease is superficial vasodilator “flush” syndrome, said composition further comprising, a flavonoid, Bitter Willow Extract, and, optionally, cyproheptadine or azatadine.

16. The composition according to claim 9, wherein said inflammatory disease is multiple sclerosis, said composition further comprising quercetin or myricetin, hydroxyzine, and, optionally, caffeine, SAMe, interferon-beta, and fragments of myelin basic protein

17. The composition according to claim 9, wherein said inflammatory disease is migraine headaches, said composition further comprising quercetin and azatadine.

18. The composition according to claim 1, said composition being for oral use, further comprising 500-1200 mg of OKE, 150-300 mg per capsule or tablet of each of chondroitin sulfate, quercetin and D-glucosamine sulfate, with, and, optionally, 100-200 mg sodium hyaluronate and/or 100 mg SAM.

19. The composition according to claim 1, said composition consisting of an ointment or cream for topical application, further comprising, in % by weight, OKE, 15; chondroitin sulfate, 5; D-glucosamine sulfate, 5; quercetin, 3; sodium hyaluronate 5; and, Bitter Willow Extract 5

20. The composition according to claim 19 supplemented by at least one of the histamine-1 receptor antagonists diphenhydramine, hydroxyzine, azelastine, azatadine or cyproheptadine, 1-5 mg %., and optionally a CRH antagonist.

21. The composition according to claim 1, said composition consisting of a mouth wash composition, comprising 500-1200 mg of OKE, chondroitin sulfate and quercetin, each 0.3-0.4 M,, and, optionally, at least one of D-glucosamine sulfate, 0.4 M and SAMe, 0.15 M, in a mouth wash vehicle.

22. The composition according to claim 1, said composition consisting of a tooth paste or oral gel, comprising, in mg %, OKE, 30-50, chondroitin sulfate, 5; quercetin, 3; and, optionally, D-glucosamine sulfate, 5, in a tooth paste vehicle.

23. The composition according to claim 1, said composition consisting of a sunscreen composition, comprising, in mg %, OKE, 30-50; chondroitin sulfate, 5; quercetin 3;; and at least one of D-glucosamine sulfate, 5, and titaniun dioxide, 5, in a sun screen vehicle.

24. The composition according to claim 1, for use in treating migraine headaches, said composition comprising, in mg, OKE, 500-1200; chondroitin sulfate, 50 ; guercetin, 100; azatadine, 4; and, optionally, a CRH antagonist.

25. The composition according to claim 1, said composition comprising, in mg, OKE, 500-1200; chondroitin sulfate, 50; quercetin, 400; hydroxyzine, 50; and, optionally, a CRH antagonist.

26. The composition according to claim 1, said composition comprising, in mg, OKE, 500-1200; chondroitin sulfate, 100; D-glucosamine sulfate, 50; quercetin, 100.

27. The composition according to claim 1, comprising, in mg %, 500-1200 mg of OKE; chondroitin sulfate, 5; D-glucosamine sulfate, 5; and, quercetin, 3.

28. The composition according to claim 1, wherein said disease is cancer and wherein said composition is designed for oral use, comprising, in mg, 500-1200 of OKE; 100 of chondroitin sulfate, 25-50 of genistein and 150-300 of quercetin.

29. The composition according to claim 1, wherein said disease is atherosclerosis with or without myocardial ischemia, comprising 500-1200 mg of OKE, 100-300 mg each of non-bovine chondroitin sulfate, quercetin, folic acid, SAMe and Bitter Willow Extract, in a vehicle for oral use.

30. The composition according to claim 1, wherein said disease is interstitial cystitis or prostatitis, said composition comprising, in mg, 500-1200 of OKE, 100-300 of chondroitin sulfate, 50-300 D-glucosamine sulfate, 100-300 of sodium hyaluronate, and 100-400 quercetin,, in a vehicle for oral or intravesicle use.

31. The composition according to claim 1, wherein said inflammatory disease is multiple sclerosis, said composition comprising, in mg, 500-1200 of OKE, 50-300 each of chondroitin sulfate, quercetin, hydroxyzine and SAMe, and, optionally, interferon-beta or fragments of myelin basic protein, in a vehicle for oral use.

32. The composition according to claim 1, said composition comprising, OKE, 30-50 mg % and, in mg, chondroitin sulfate 500; myricetin 300; and diphenhydramine, 5 mg %.

33. The composition according to claim 1, said composition comprising, in mg, OKE, 900-1200; chondroitin sulfate, 50; kaempferol, 100; SAMe, 50; folic acid, 50; and, niacin, 100.

34. The composition according to claim 1, wherein said disease is superficial vasodilation flush syndrome, said composition comprising 500-900 mg OKE; 50 mg chondroitin sulfate; 150 mg quercetin; 5% by weight Bitter Willow Extract, and, optionally, 4 mg cyproheptadine or azatadine.

35. The composition according to claim 1, wherein said disease is skin allergy, said composition comprising, in % by weight, 15 of OKE, 5 each of aloe vera, chondroitin sulfate and alpha-tocopherol, and, optionally, azelastine.

36. The composition according to claim 1, wherein said disease in allergy or allergic asthma, comprising 500-1200 of OKE; 500 mg of myricetin; 200 mg of chondroitin sulfate; and, optionally, azelastine or hydroxyzine.

37. The composition according to claim 36, in an aerosol vehicle.

38. The composition according to claim 1, wherein said disease is a hormonally-dependent cancer, comprising, in mg, 500-1200 OKE; 150 each of chondroitin sulfate and quercetin; 50 genestein; and, optionally, 10 tamoxifen or raloxifen, in an oral vehicle.

39. The composition according to claim 1, wherein said disease is allergic conjunctivitis, comprising in mg %, OKE 30-50; quercetin 0.05; chondroitin sulfate 2.0; and, optionally, azelastine 0.05, in an ocular vehicle.

40. The composition according to claim 1, wherein in said disease is spontaneous recurrent pregnancy loss, and said composition comprises, in mg %, OKE 500-1200; quercetin 500; chondroitin sulfate 200; and, optionally, a CRH antagonist, in an intravaginal vehicle.
